(19) United States
(12) Patent Application Publication
(10) Pub. No.: US 2020/0279657 A1
(43) Pub. Date: Sep. 3, 2020

(54) INFECTIOUS DISEASE PROLIFERATION PREVENTION SYSTEM AND METHOD

(71) Applicant: KT CORPORATION, Seongnam-si (KR)

(72) Inventors: Jae-Ho LEE, Seoul, (KR); Joon-Ki CHOI, Seoul (KR)

(73) Assignee: KT CORPORATION, Seongnam-si (KR)

(21) Appl. No.: 16/643,370

(22) PCT Filed: Aug. 20, 2018
(86) PCT No.: PCT/KR2018/009545

§ 371 Date: May 11, 2020

(30) Foreign Application Priority Data
Aug. 31, 2017 (KR) 10-2017-0111407
Dec. 18, 2017 (KR) 10-2017-0174345

(51) Int. Cl. G16H 50/80; H04W 4/029; H04W 64/00; H04W 4/12; G06F 21/62

(52) U.S. Cl. CPC G16H 50/80; H04W 4/029; G06F 21/6245; H04W 4/12; H04W 64/00

(57) ABSTRACT

Disclosed is system for preventing proliferation of an infectious disease comprising: at least one processor; and a memory including at least one command in which at least one processor can be executed, wherein at least one processor: collects infectious disease information; receives consent for a collection and use of personal information from a service user which wants to use an infectious disease proliferation prevention service; transmits, to a plurality of mobile communication systems, the infectious disease information and the consent for a collection and use of personal information; receives, from the plurality of mobile communication systems, information on a mobile terminal which has visited a contaminated area, calculated on the basis of the infectious disease information, the consent for a collection and use of personal information and wireless communication information of the mobile terminal; and classifies, by infectious diseases, and manages the information on a mobile terminal which has visited a contaminated area, received from the plurality of mobile communication systems.

### CROSS-REFERENCE TO RELATED APPLICATIONS

This is a national stage application of PCT/KR2018/009545 filed on Aug. 20, 2018 which claims priority from Korean Patent Application No. 10-2017-0111407 filed on Aug. 31, 2017, in the Korean Intellectual Property Office, the disclosures of which are hereby incorporated herein in their entirety by reference.

### BACKGROUND

### 1. Field

The present disclosure relates to preventing the spread of infectious diseases, and more particularly, to an infectious disease proliferation prevention system and method for preventing infectious diseases in some areas from spreading across the world using wireless communication information of mobile terminals possessed by global communication companies in the world.

### 2. Related Art

The global challenge is the fast spread of endemic infectious diseases (hereinafter, infectious diseases), for example, Ebola, Middle East respiratory syndrome (MERS) and Zika virus diseases. In 2015, the Republic of Korea had the MERS national disaster. Prior to that, in 2014, there were about 11,000 deaths around the world since the Ebola outbreak. In 2016, Zika viruses have spread to 29 countries in two months, causing losses of about 64 billion dollars in the tourism industry. Additionally, it is foreseen that at least one infectious disease will sweep across the world within 100 years in the future and the probability that at least four infectious diseases will occur is 20% or higher. Additionally, it is foreseen that global average economic losses of 60 billion dollars or more per year will occur due to potential infectious diseases.

Meanwhile, infectious diseases in some areas tend to spread across the world rapidly and widely. The reason is as follows: the advent of many low-cost airlines makes it easy to move from place to place all over the world. Additionally, due to income rises, changes in attitude towards life, and emergence of the concept of YOLO or you only live once, the number of people who move abroad for the purpose of trip and business is increasing at a fast pace. In particular, Africa, India and South America were least-visited countries, but as there is an increase in the number of travelers to these areas, fatal infectious diseases in there are more likely to spread across the world.

If it fails to prevent infection in infectious disease-affected areas, infectious diseases will spread all over the world, causing tremendous damage. In 2015, MERS was introduced to the Republic of Korea by a patient returning from Saudi Arabia, and then, the patient was not screened at the airport, and after symptoms developed, the first visited hospital failed to prevent infection. As a result, since the MERS outbreak, epidemiology and prevention efforts have been devoted so much, but nevertheless tremendous damage occurred. To prevent the spread of infectious diseases, global efforts are required to manage the infectious diseases.

As part of these efforts, most countries force, by law, people returning from infectious disease-affected areas to report their visit to the infectious disease-affected areas. However, to report the visit to infectious disease-affected areas, visitors should know information about the types of the infectious diseases and the infectious disease-affected areas. However, there are many types of infectious diseases, and the infectious disease-affected areas may change over time, so it is difficult to create environments in which visitors can make voluntary reports. Additionally, in case that reporting is not mandatory and is done voluntarily as a personal duty, if damage occurs afterwards, that will be so much.

Accordingly, there is a need for global efforts to accurately detect people who have visited infectious disease-affected areas, and provide them with testing services related to the corresponding infectious diseases.

### SUMMARY

The present disclosure relates to an infectious disease proliferation prevention system and method for preventing an infectious disease occurred in a certain area from spreading to other areas using infectious disease information occurring everywhere in the world and wireless communication information of mobile terminals in the world.

These and other objects and advantages of the present disclosure may be understood by the following description and will be apparent from the embodiments of the present disclosure. In addition, it will be readily understood that the objects and advantages of the present disclosure may be realized by the means set forth in the appended claims and a combination thereof.

A system for preventing the spread of an infectious disease according to an aspect includes at least one processor, and a memory including at least one command that the at least one processor can execute, wherein the at least one processor collects infectious disease information, receives consent to collection and use of personal information from a service user who wants to use a service for preventing the spread of an infectious disease, transmits the infectious disease information and the consent to collection and use of personal information to a plurality of mobile communication systems, receives, from the plurality of mobile communication systems, information of a mobile terminal having visited an affected area, wherein the information of a mobile terminal is calculated based on the infectious disease information, the consent to collection and use of personal information and wireless communication information of the mobile terminal, and classifies and manages the information on the mobile terminal having visited the affected area received from the plurality of mobile communication systems by infectious disease.

The at least one processor may receive missing terminal information based on the consent to collection and use of personal information from each of the plurality of mobile communication systems, collect the missing terminal information, generate a missing terminal list, transmit the missing terminal list to the plurality of mobile communication systems, and receive the information of the mobile terminal having visited the affected area from the plurality of mobile communication systems in response.

The missing terminal information may be information about a mobile terminal that has consented to collect and use personal information, but has not connected to a corresponding network for a preset period or has an international roaming service history among mobile terminals subscribed to each mobile communication system.

The information of the mobile terminal having visited the affected area may be information about a mobile terminal that has a history of communication with a base station that matches the infectious disease information, and is included in the missing terminal list.

The at least one processor may classify the information of the mobile terminal having visited the affected area, received from the plurality of mobile communication systems by infectious disease, and generate and store a list, and the list classified by infectious disease may include at least one of identification information of the mobile terminal having visited the affected area, an infectious disease name, a first visit date, a last visit date, and number of days left for an incubation period expires.

Each time the at least one processor receives the information of the mobile terminal having visited the affected area from the plurality of mobile communication systems, the at least one processor may update the last visit date and the number of days left until the incubation period expires for the corresponding mobile terminal in the list classified by infectious disease.

The at least one processor may transmit a message notifying expiration of the incubation period of the infectious disease to the mobile terminal with the expired incubation period of the infectious disease based on the list classified by infectious disease.

The at least one processor may transmit the infectious disease information, and the consent to collection and use of personal information of a service user having subscribed to a corresponding mobile communication system, except a service user having subscribed to other mobile communication system, to each of the plurality of mobile communication systems.

The infectious disease information may include at least one of an infectious disease name, date of infectious disease outbreak, date of infectious disease eradication, a name of an affected country, and a name of an affected area.

The at least one processor may transmit a message notifying an entry into the affected area to the mobile terminal having visited the affected area.

A method for preventing the spread of an infectious disease by a system for preventing the spread of an infectious disease according to another aspect includes collecting infectious disease information, receiving consent to collection and use of personal information from a service user who wants to use a service for preventing the spread of an infectious disease, transmitting the infectious disease information and the consent to collection and use of personal information to a plurality of mobile communication systems, receiving information of a mobile terminal having visited an affected area, wherein the information of a mobile terminal is calculated based on the infectious disease information, the consent to collection and use of personal information and wireless communication information of the mobile terminal from the plurality of mobile communication systems, and classifying and managing the information of the mobile terminal having visited the affected area, received from the plurality of mobile communication companies by infectious disease.

Receiving the information of the mobile terminal having visited the affected area may include receiving missing terminal information based on the consent to collection and use of personal information from each of the plurality of mobile communication systems, collecting the missing terminal information, generating a missing terminal list, transmitting the missing terminal list to the plurality of mobile communication systems, and receiving the information of the mobile terminal having visited the affected area from the plurality of mobile communication systems in response.

The missing terminal information may be information about a mobile terminal that has consented to collection and use of personal information among mobile terminals subscribed to each mobile communication system, but has not connected to a corresponding network for a preset period or has an international roaming service history.

The information of the mobile terminal having visited the affected area may be information of a mobile terminal that has a history of communication with a base station matching the infectious disease information and is included in the missing terminal list.

Classifying and managing may include classifying the information of the mobile terminal having visited the affected area received from the plurality of mobile communication systems by infectious disease and generating and storing a list, and the list classified by infectious disease may include at least one of identification information of the mobile terminal having visited the affected area, an infectious disease name, a first visit date, a last visit date, and number of days left until an incubation period expires.

The method may further include, updating the last visit date and the number of days left until the incubation period expires for the corresponding mobile terminal in the list classified by infectious disease, each time the information of the mobile terminal having visited the affected area is received from the plurality of mobile communication systems.

The method may further include transmitting a message notifying expiration of the incubation period of the infectious disease to the mobile terminal with the expired incubation period of the infectious disease based on the list classified by infectious disease.

Transmitting the consent to collection and use of personal information may include transmitting the infectious disease information and the consent to collection and use of personal information of a service user having subscribed to a corresponding mobile communication system, except a service user having subscribed to other mobile communication system, to each of the plurality of mobile communication systems.

The infectious disease information may include at least one of an infectious disease name, date of infectious disease outbreak, date of infectious disease eradication, a name of an affected country, and a name of an affected area.

The method may further include transmitting a message notifying an entry into the affected area to the mobile terminal having visited the affected area.

According to an embodiment, it is possible to prevent infectious diseases occurred in some areas from spreading to other areas by accurately detecting people who have visited infectious disease-affected areas using wireless communication information of mobile terminals and taking a variety of subsequent actions.

According to an embodiment, it is possible to provide related information to people who have visited infectious disease-affected areas to advise to take caution of the infectious diseases in order to avoid or prevent the exposure to the infectious diseases.

### BRIEF DESCRIPTION OF DRAWINGS

FIG. 1 is a diagram showing a communication environment of an infectious disease proliferation prevention system according to an embodiment.

FIG. 2 is a block diagram showing the architecture of the infectious disease proliferation prevention system of FIG. 1.

FIG. 3 is a diagram showing information transmitted by an infectious disease proliferation prevention system for each mobile communication system according to an embodiment.

FIG. 4 is a diagram showing missing terminal information transmitted to an infectious disease proliferation prevention system by each mobile communication system according to an embodiment.

FIG. 5 is a diagram showing a list of all missing terminals prepared by an infectious disease proliferation prevention system according to an embodiment.

FIG. 6 is a diagram showing information of affected area visitor mobile terminals transmitted to an infectious disease proliferation prevention system by each mobile communication system according to an embodiment.

FIG. 7 is a diagram showing a list of all affected area visitor mobile terminals prepared by an infectious disease proliferation prevention system according to an embodiment.

FIG. 8 is a flowchart illustrating an infectious disease proliferation prevention method according to an embodiment.

FIG. 9 is a detailed flowchart illustrating a step of receiving affected area visitor terminal information of FIG. 8.

### DETAILED DESCRIPTION OF EXEMPLARY EMBODIMENT

The foregoing objects, features, and advantages will become apparent from the following detailed description with reference to the accompanying drawings, and accordingly, those skilled in the art will be able to easily practice the technical aspects of the present disclosure. Furthermore, in the description of the present disclosure, when it is deemed that a certain detailed description of known technology related to the present disclosure may unnecessarily render the key subject matter of the present disclosure ambiguous, the detailed description is omitted herein. Hereinafter, an exemplary embodiment of the present disclosure will be described in detail with reference to the accompanying drawings.

It is possible to monitor travelers' visits to specific countries using airport entry and exit information. Additionally, it is possible to identify travelers' visits to infectious disease-affected countries and areas using credit card payment information from financial companies and reservation information from travel agencies/insurance companies/airlines. It is possible to identify countries and areas information that travelers visited through credit card payment information from credit card companies or bank exchange information in foreign countries. Additionally, it is possible to identify foreign countries and areas that travelers visited using flight and hotel booking information from travel agencies and traveler insurance information. However, in the case of credit cards, it is possible to identify travelers' visits to infectious disease-affected countries only when the travelers use their credit cards in the corresponding countries. It is difficult to manage even lots of medium- and small-sized travel agencies.

Many mobile communication subscribers use international roaming services when visiting foreign countries. Some mobile communication subscribers buy and use Subscriber Identity Module (SIM) cards in foreign countries that they visited. Mobile communication companies can detect countries that their subscribers visited while international roaming, but cannot know the detailed information about cities or areas in the corresponding countries. Additionally, when their subscribers buy and use SIM cards in foreign counties, the mobile communication companies cannot even know the countries that the subscribers visited. Meanwhile, no matter whether mobile communication subscribers use international roaming or local SIM cards, the subscribers' mobile terminals use wireless communication services (for example, voice call, video call, text message transmission/reception, wireless Internet) via base stations covering the areas in which the subscribers are currently located. Accordingly, local mobile communication companies that manage the corresponding base stations can detect area information where the mobile terminals are located. Accordingly, cooperation between mobile communication companies in the world makes it possible to detect countries and even areas that mobile communication subscribers visited using wireless communication information of mobile terminals. An embodiment of the present disclosure detects countries that mobile communication subscribers visited and the period of stay using wireless communication information of mobile communication systems. Using the wireless communication information, it is possible to detect countries and areas subscribers visited and the period of stay. When infectious disease outbreak information is applied to it, it is possible to analyze visits to infectious disease-affected countries or areas and infectious disease risks for each person.

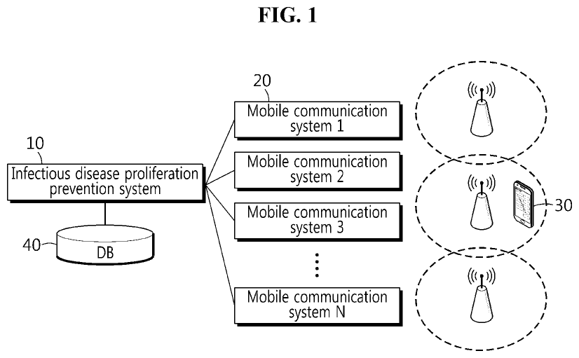

FIG. 1 is a diagram showing a communication environment of an infectious disease proliferation prevention system according to an embodiment. Referring to FIG. 1, the infectious disease proliferation prevention system 10 forms an alliance with a plurality of mobile communication companies in the world to communicate with mobile communication systems 20:1, 2, 3, . . . , N of the plurality of mobile communication companies. The infectious disease proliferation prevention system 10 stores and manages infectious disease information occurred in the world, and continuously manages infectious disease-affected area visitors extracted based on wireless communication information collected by the mobile communication systems 20 of the plurality of mobile communication companies, to prevent the spread of the infectious diseases across the world.

The mobile communication system 20 provides a wireless communication service to a mobile terminal 30 subscribed to the corresponding company, a mobile terminal 30 allowed to roam abroad, or a mobile terminal 30 of a foreign traveler who buys and uses a SIM card in a country that the traveler visited. The mobile communication system 20 may extract information of a mobile terminal 30 having actually visited the infectious disease-affected area by matching the infectious disease information received from the infectious disease proliferation prevention system 10 and a list of mobile terminals suspected to visit infectious disease-affected areas, with the wireless communication information of the mobile terminal 30, and transmit the extracted information of the mobile terminal 30 to the infectious disease proliferation prevention system 10. To provide the wireless communication service to the mobile terminal 30, the mobile communication system 20 periodically detects location information of the mobile terminals 30, and automatically collects the wireless communication information of the mobile terminal 30 when the mobile terminal 30 is powered on. Here, the wireless communication information may include area information (location information of the mobile terminal) that the user of the mobile terminal 30 visited, the first visit date and the last departure date, and identification information (for example, International Mobile Equipment Identity (IMEI), International Mobile Subscriber Identity (IMSI), Mobile Subscriber Integrated Service Digital Network (MSISDN)) of the mobile terminal.

When the mobile terminal 30 subscribed to the corresponding company go abroad and use an international roaming service, the mobile communication system 20 may collect roaming data of the corresponding mobile terminal 30. For example, when the mobile terminal 30 performs location registration in the foreign visited network (for example, power on when arriving at the country that the mobile terminal 30 visited), the mobile communication system 20 may collect the roaming data of the mobile terminal 30. The roaming data is information of the visited network, and includes communication company information of the visited network and country information. For example, when the user of the mobile terminal 30 takes a trip to Kenya, Africa and uses an international roaming service, the mobile communication system 20 of the home network to which the mobile terminal 30 has subscribed, for example, Republic of Korea KT, may detect that the visited network that the user of the mobile terminal 30 visited is in Kenya using roaming data. The mobile communication system 20 of the home network cannot collect information about an area that the user of the corresponding mobile terminal 30 visited in Kenya. In contrast, the mobile communication system 20 of the visited network that provides the wireless communication service in Kenya, for example, Safaricom, may collect information (visited area information) about an area that the user of the corresponding mobile terminal 30 visited near a popular restaurant in Nairobi (capital), Kenya, while providing the wireless communication service to the corresponding the mobile terminal 30. Additionally, the mobile communication system 20 of the home network may analyze countries that the mobile terminal 30 visited through analysis of transmitted/received text message records, or voice or video call records of the mobile terminal 30 in the foreign visited network.

The infectious disease proliferation prevention system 10 may provide an infectious disease proliferation prevention service using the wireless communication information of the mobile terminal 30 possessed by the plurality of mobile communication systems 20 without directly collecting the wireless communication information of the mobile terminal 30. In an embodiment, the mobile communication system 20 may refer to a single network equipment or a set of network equipment that provides a mobile communication service, and performs an operation for infectious disease proliferation prevention by collecting wireless communication information for infectious disease proliferation prevention and communicating with the infectious disease proliferation prevention system 10.

The mobile terminal 30 is a mobile communication terminal, and is a device that can use message services, call services or wireless Internet services by accessing the mobile communication system 20. When the mobile terminal 30 moves from the home network to the visited network or from the visited network to the home network, the mobile terminal 30 may perform location registration, and the mobile communication system 20 may detect the country at which the mobile terminal 30 is currently located using location information at the time of location registration. The mobile terminal 30 may be used interchangeably with a User Equipment (UE), a Mobile Station (MS), a User Terminal (UT), a Subscriber Station (SS), or the like.

A database 40 may store information necessary to provide the infectious disease proliferation prevention service, and provide information requested from the infectious disease proliferation prevention system 10. According to an embodiment, the database 40 may store a list of mobile communication systems 20:1, 2, 3, . . . , N by country, infectious disease information occurred in the world updated in real time, and a list of mobile terminals 30 having visited affected areas.

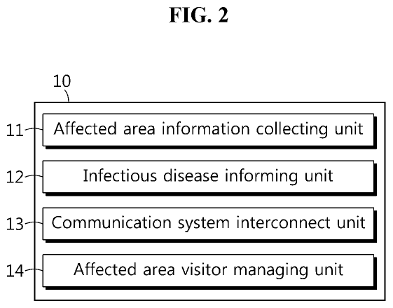

FIG. 2 is a block diagram showing the architecture of the infectious disease proliferation prevention system 10 of FIG. 1. The infectious disease proliferation prevention system 10 may include a memory, a memory controller, at least one processor (CPU), a peripheral interface, an input/output (I/O) subsystem, a display device, an input device and a communication circuit. These components communicate through at least one communication bus or signal line. These components include at least one signal processor and/or application specific integrated circuit, and may be implemented in either hardware or software or in combination.

The memory may include a high speed random access memory, and may include at least one magnetic disk storage device, nonvolatile memory such as a flash memory device, or other nonvolatile semiconductor memory device. In some embodiments, the memory may further include a storage device located far away from the at least one processor, for example, a RF circuit and a network attached storage device that is accessed through a communication network (not shown) such as Internet, Intranet, Local Area Network (LAN), Wide LAN (WLAN), Storage Area Network (SAN) or a proper combination thereof. The access to the memory by other component of the device such as the processor and the peripheral interface may be controlled by the memory controller.

The peripheral interface connects the I/O peripheral device of the system to the processor and memory. The at least one processor performs many functions for the system by executing various software programs and/or an instruction set stored in the memory and performs data processing. In some embodiments, the peripheral interface, the processor and the memory controller may be implemented on a single chip such as a chip. In other embodiments, the peripheral interface, the processor and the memory controller may be implemented as different chips.

The I/O subsystem provides interface between the I/O peripheral device of the device such as the display device and the input device and the peripheral interface.

The display device may use liquid crystal display (LCD) technology or light emitting polymer display (LPD) technology, and the display device may include capacitive, resistive and infrared touch displays. The touch display provides output interface and input interface between the system and the user. The touch display displays a visual output to the user. The visual output may include text, graphics, video and a combination thereof. The visual output may conform to a target for user interface in whole or in part. The touch display forms a touch sensing surface for receiving the user input.

The processor is configured to perform the operation associated with the system and execute the instructions, and may control the reception and manipulation of input and output data between the components of the system, for example, using the instructions retrieved from the memory.

In some embodiments, the software components include an operating system (OS), a graphics module (an instruction set), and a program for performing the operation for the present disclosure mounted (installed) in the memory. The OS may be an embedded OS such as, for example, Darwin, RTXC, LINUX, UNIX, OSX, WINDOWS or VxWorks, Android and iOS, and includes various software components and/or devices to control and manage a general system task (for example, memory management, storage device control, power management), and promotes the communication between various hardware and software components. The graphics module includes known software components for providing and displaying graphics on the display device. The term “graphics” includes, without limitation, text, a web page, an icon (for example, a target for user interface including a soft key), digital image, video, animation, and includes any type that can be displayed to the user.

The communication circuit may include an Ethernet communication circuit and a RF circuit. The Ethernet communication circuit performs wired communication, and the RF circuit transmits and receives electromagnetic waves. The RF circuit converts electrical signals to electromagnetic waves or vice versa, and communicates with a communication network, a mobile gateway and a communication device through the electromagnetic waves. The RF circuit may include, for example, an antenna system, a RF transceiver, at least one amplifier, a tuner, at least one oscillator, a digital signal processor, a CODEC chipset and a memory, but is not limited thereto, and may include known circuits for performing these functions. The RF circuit may communicate with other device by a wireless network such as a cellular phone network, wireless LAN and/or a metropolitan area network (MAN), and short range wireless communication.

Referring to FIG. 2, the infectious disease proliferation prevention system 10 may include an affected area information collecting unit 11, an infectious disease informing unit 12, a communication system interconnect unit 13 and an affected area visitor managing unit 14. The affected area information collecting unit 11, the infectious disease informing unit 12, the communication system interconnect unit 13 and the affected area visitor managing unit 14 may be implemented in software, stored in the memory, and performed by a processor, or may be implemented in the form of a module of software and hardware in combination and performed.

The affected area information collecting unit 11 may collect infectious disease information from a variety of Internet sites that provide infectious disease information occurred in the world and store the infectious disease information in the database 40. For example, the affected area information collecting unit 11 may crawl infectious disease information on sites such as WHO IHR, Promed, GPHIN and store the infectious disease information in the database 40. Here, the infectious disease information may include at least one of an infectious disease name, the date of infectious disease outbreak, the date of infectious disease eradication, the name of an affected country, and the name of an affected area.

According to an embodiment, the information stored in the database 40 is transmitted to the mobile communication system 20 in each country and used as data for location based analysis, and the affected area information collecting unit 11 may convert TEXT information of the area into a format for Geographic Information System (GIS) analysis, and additionally store it. Its example is shown in the following table 1.

TABLE 1

| | | Date of | Date of | | | | |
|---|---|---|---|---|---|---|---|
| | infectious | infectious | infectious | Name of | Name of | | |
| Name of | disease | disease | disease | affected | affected | | |
| field | name | outbreak | eradication | country | area | Type | Availability |
| Format | TEXT | POSIXct | POSIXct | TEXT | TEXT | TEXT | Boolean |

The infectious disease informing unit 12 may receive consent to collection and use of personal information from a person (hereinafter, a service user) who wants to use the infectious disease proliferation prevention service, and collect identification information (for example, IMEI) of the mobile terminal 30 of the service user and information of the mobile communication system 20 to which the service user subscribed. Here, the consent to collection and use of personal information may include consent to information related to the purpose of collection and use of personal information (for example, providing an advice service about visit to an infectious disease-affected area), information to use (for example, wireless communication information between the mobile terminal and the base station, collected by the mobile communication system), the period of collection and use of personal information (for example, equal to the incubation period of an occurred infectious disease), and personal information deletion (for example, deleting when cancelling the service).

The infectious disease informing unit 12 may provide infectious disease related information to the service user. When the service user goes into the infectious disease-affected area for the first time, or within the incubation period of the infectious disease, or at the end of the incubation period of the infectious disease, the infectious disease informing unit 12 may transmit an infectious disease related message appropriate for each situation to the mobile terminal 30 of the service user based on the analysis result of the affected area visitor managing unit 14. The infectious disease related message may be a short message service (SMS) message or a multimedia messaging service (MMS) message.

The communication system interconnect unit 13 is connected to the plurality of mobile communication systems 20 in the world to transmit and receive data. The communication system interconnect unit 13 may transmit the infectious disease information collected by the affected area information collecting unit 11 and the consent to collection and use of personal information collected by the infectious disease informing unit 12 to the plurality of mobile communication systems 20. Additionally, the communication system interconnect unit 13 may receive information of a mobile terminal having visited an affected area (hereinafter referred to as ‘affected area visitor terminal information’) from the plurality of mobile communication systems 20. The communication system interconnect unit 13 may identify the mobile communication system 20 to which each of a plurality of service users subscribed by referencing the database 40, classify the service users for each mobile communication system 20, and transmit the infectious disease information, the identification information (for example, IMEI) of the mobile terminal 30 of the service user and the consent to collection and use of personal information to each mobile communication system 20.

The communication system interconnect unit 13 may manage the plurality of mobile communication systems 20 in the world by unique numbers, and the unique number may include code defined by ITU-T Recommendation E.212. The code includes a combination of 3-digit country code and 3-digit business code, and the communication system interconnect unit 13 may manage the plurality of mobile communication systems 20 in the world by affixing information about interconnect next to the code. According to an embodiment, the communication system interconnect unit 13 may classify the infectious disease information collected by the affected area information collecting unit 11 by country, and only provide the infectious disease information occurred in the service areas of each mobile communication system 20 to the corresponding mobile communication systems 20 respectively.

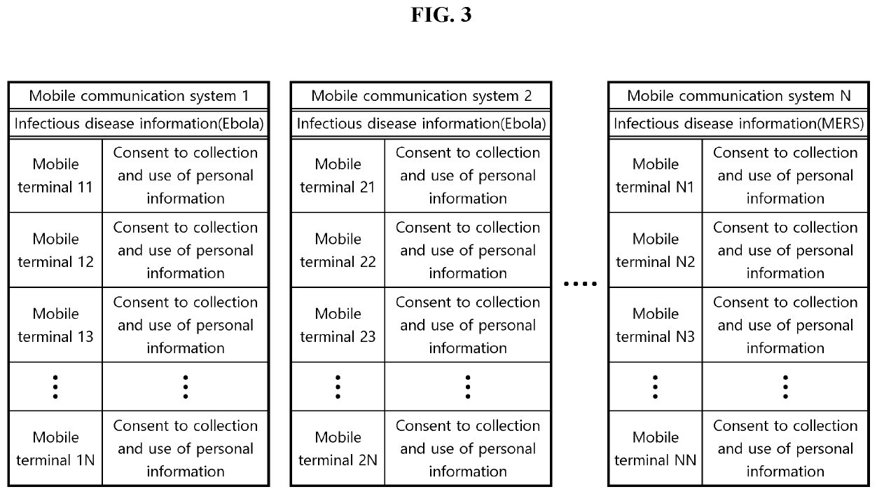

FIG. 3 is a diagram showing information transmitted by the infectious disease proliferation prevention system for each mobile communication system according to an embodiment. Referring to FIG. 3, for example, the communication system interconnect unit 13 may classify a plurality of mobile terminals 11, 12, 13, . . . , 1N subscribed to a first mobile communication system 1, a plurality of mobile terminals 21, 22, 23, . . . , 2N subscribed to a second mobile communication system 2, and a plurality of mobile terminals N1, N2, N3, . . . , NN subscribed to an Nth mobile communication system N, and transmit each of infectious disease information occurred in service areas of each mobile communication system 20, identification information (for example, IMEI, IMSI) of the mobile terminals 30 subscribed to each mobile communication system 20 and consent to collection and use of personal information. Accordingly, the communication system interconnect unit 13 may individually transmit the identification information (for example, IMEI, IMSI) of the mobile terminals 30 of the service users and the consent to collection of use of personal information for each mobile communication system 20, thereby solving the security problem and the resource waste problem that may occur when consent to collection and use of personal information of all the service users in the world and identification information (for example, IMEI, IMSI) of the mobile terminals 30 are all equally transmitted to the plurality of mobile communication systems 20.

Each of the plurality of mobile communication systems 20 having received the consent to collection and use of personal information from the communication system interconnect unit 13 may extract mobile terminals 30 having consented to collect and use personal information from the mobile terminals subscribed to the corresponding mobile communication system 20, and determine, as missing terminals, mobile terminals 30 that have not connected to the corresponding network for a preset period or have an international roaming service history among the extracted mobile terminals 30.

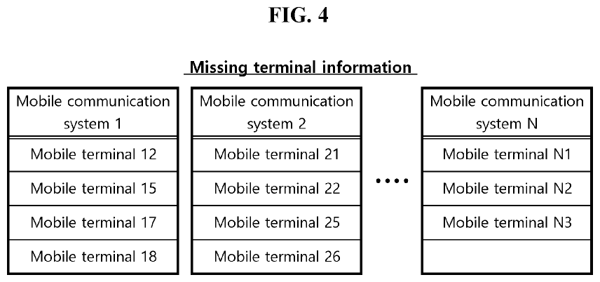

FIG. 4 is a diagram showing missing terminal information transmitted to the infectious disease proliferation prevention system by each mobile communication system according to an embodiment. Referring to FIGS. 3 and 4, for example, the first mobile communication system 1 determine, as missing terminals 12, 15, 17, 18, mobile terminals 30 that have not connected to the corresponding network for the preset period or have an international roaming service history among the plurality of mobile terminals 11, 12, 13, . . . , 1N received from the communication system interconnect unit 13. The second mobile communication system 2 and the Nth mobile communication system N may determine missing terminals 21, 22, 25, 26 and missing terminals N1, N2, N3 respectively by the same method. Subsequently, the first, second and Nth mobile communication systems 1, 2, N may transmit missing terminal information including identification information (for example, IMEI) of the missing terminals to the communication system interconnect unit 13.

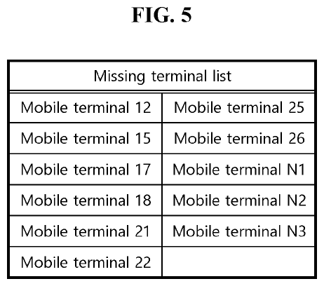

FIG. 5 is a diagram showing a list of all missing terminals prepared by the infectious disease proliferation prevention system using the collected missing terminal information received from the plurality of mobile communication systems according to an embodiment. Referring to FIG. 5, the communication system interconnect unit 13 may receive the missing terminal information from each mobile communication system 20 and generate a missing terminal list. For example, as shown in FIG. 4, the communication system interconnect unit 13 may individually receive the missing terminal information from the first, second and Nth mobile communication systems 1, 2, N, collect the missing terminal information, and generate the missing terminal list shown in FIG. 5. The generated missing terminal list may be shared among the first, second and Nth mobile communication systems 1, 2, N.

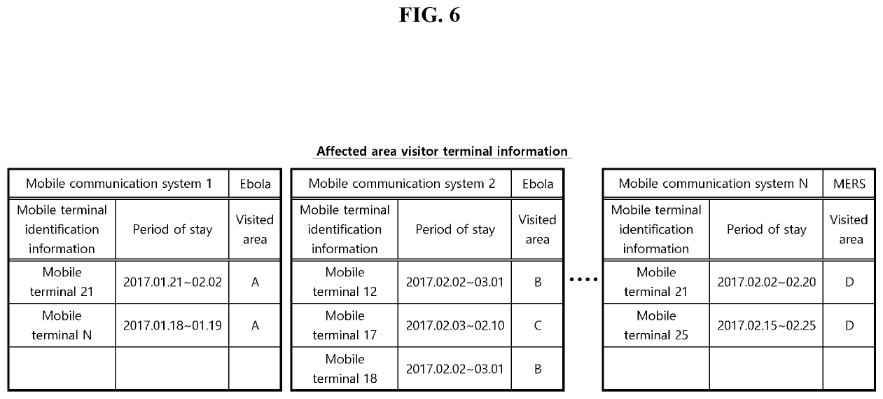

The communication system interconnect unit 13 may transmit the missing terminal list to the plurality of mobile communication systems 20, and receive affected area visitor terminal information from the plurality of mobile communication systems 20. In this instance, each of the plurality of mobile communication systems 20 may extract base stations installed in affected areas by matching information about the infectious disease affected areas received from the communication system interconnect unit 13 with information of the base stations managed by the corresponding company, and extract mobile terminals 30 having a history of communication with the extracted base stations. Each of the plurality of mobile communication systems 20 may determine a mobile terminal 30 included in the missing terminal list among the extracted mobile terminals as an affected area visitor terminal, and transmit affected area visitor terminal information including identification information (for example, IMEI) of the affected area visitor terminal, the visited infectious disease affected area, the name of the infectious disease and the period of stay to the communication system interconnect unit 13. FIG. 6 is a diagram showing the affected area visitor terminal information transmitted to the infectious disease proliferation prevention system 10 by each mobile communication system 20 according to an embodiment.

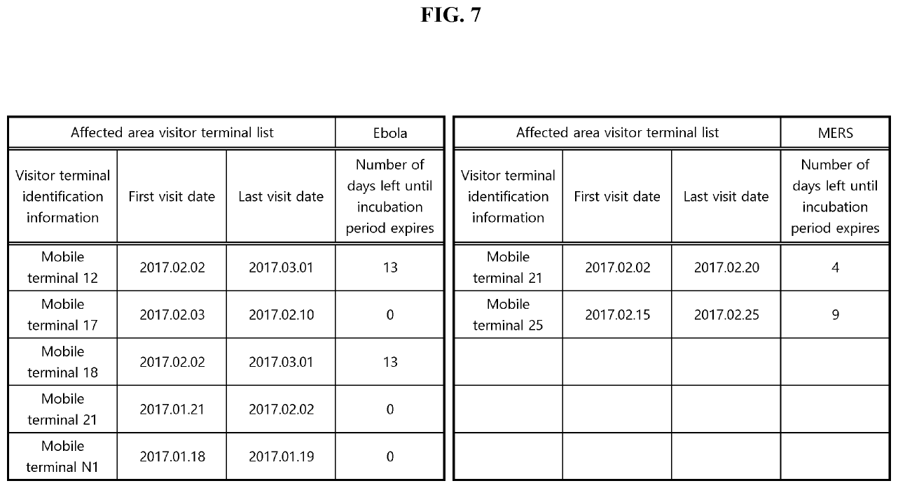

The affected area visitor managing unit 14 may classify the affected area visitor terminal information received from the plurality of mobile communication systems 20 by infectious disease, generate an affected area visitor terminal list by infectious disease and store the affected area visitor terminal lists in the database 40. FIG. 7 is a diagram showing a list of all affected area visitor terminals prepared by the infectious disease proliferation prevention system after collecting affected area visitor terminal information received from a plurality of mobile communication systems according to an embodiment. Referring to FIGS. 6 and 7, the affected area visitor managing unit 14 may collect the affected area visitor terminal information as shown in FIG. 6 transmitted to the communication system interconnect unit 13 by the mobile communication systems 1, 2, N, and generate the affected area visitor terminal list by infectious disease as shown in FIG. 7. Here, the affected area visitor terminal list may include the items of identification information (for example, IMEI) of the affected area visitor terminal, the name of the infectious disease, the first visit date, the last visit date and the number of days left until the incubation period expires.

According to an embodiment, the affected area visitor managing unit 14 may manage infectious disease-affected area visitors through the affected area visitor terminal list by adding, updating and deleting the affected area visitor terminal information received from the plurality of mobile communication systems 20 in a predetermined cycle.

When the affected area visitor terminal is transmitted for the first time and thus there is no record stored in the database 40, the affected area visitor managing unit 14 may add and store the corresponding affected area visitor terminal information to the affected area visitor terminal list. In this instance, when the affected area visitor terminal is added to the affected area visitor terminal list for the first time, the infectious disease informing unit 12 may transmit the fact that the affected area visitor terminal entered the affected area to the affected area visitor terminal. The infectious disease informing unit 12 may transmit an infectious disease related message about the name of the infectious disease occurred in the affected area, symptoms of the corresponding infectious disease and actions to be taken when symptoms develop, to the affected area visitor terminal in the form of an SMS message, an MMS message or a Push message of smartphone Application.

When there is a record of the affected area visitor terminal stored in the database 40, first, the affected area visitor managing unit 14 may identify if the infectious disease name is the same, and when the infectious disease name is the same, may update the affected area visitor terminal list by changing the last visit date to the recently received date, and re-calculating the number of days left until the incubation period expires. In this instance, the infectious disease informing unit 12 may transmit information about the corresponding infectious disease to the affected area visitor terminal in the form of an SMS message or an MMS message within the incubation period. When the infectious disease name is different for the same affected area visitor terminal, the affected area visitor managing unit 14 may determine the exposure to a new infectious disease, generate data together with the name of the infectious disease, and add the data to the affected area visitor terminal list for the corresponding infectious disease.

The affected area visitor managing unit 14 may update the affected area visitor terminal list in the predetermined cycle, and when the affected area visitor terminal list is updated, may calculate affected area visitor terminal information in which the incubation period is determined to expire and delete the affected area visitor terminal information from the affected area visitor terminal list.

FIG. 7 shows the calculation of affected area visitor terminal information in which the incubation period is determined to expire by the affected area visitor managing unit 14 under the assumption that the current date is Mar. 2, 2017. For example, referring to FIG. 7, in the case of the mobile terminal 17, when a result (for example, Feb. 24, 2017) of adding the last visit date (for example, Feb. 10, 2017) to the incubation period (14 days) in the recently updated affected area visitor terminal list is earlier than the current date (Mar. 2, 2017), the affected area visitor managing unit 14 may determine that the incubation period expired (the number of days left until the incubation period expires=0), and delete the mobile terminal 17 from the affected area visitor terminal list. When the affected area visitor terminal is deleted from the affected area visitor terminal list, the infectious disease informing unit 12 may inform the expiration of the incubation period to the affected area visitor terminal in the form of an SMS message or an MMS message.

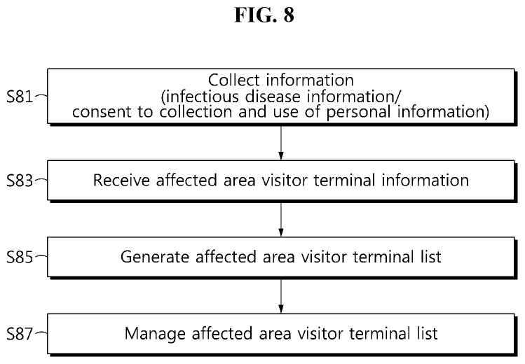

FIG. 8 is a flowchart illustrating an infectious disease proliferation prevention method according to an embodiment. Referring to FIG. 8, the infectious disease proliferation prevention method may include collecting information (S81), receiving affected area visitor terminal information (S83), generating an affected area visitor terminal list (S85), and managing the affected area visitor terminal list (S87).

In S81, the infectious disease proliferation prevention system 10 may collect infectious disease information occurred in the world from Internet sites that provide infectious disease information and store the infectious disease information in the database 40. For example, the infectious disease proliferation prevention system 10 may crawl infectious disease information on sites such as WHO IHR, Promed, GPHIN and store the infectious disease information in the database 40. Here, the infectious disease information may include at least one of the infectious disease name, the date of infectious disease outbreak, the date of infectious disease eradication, the name of an affected country, and the name of an affected area.

Additionally, the infectious disease proliferation prevention system 10 may receive consent to collection and use of personal information from a service user who wants to use an infectious disease proliferation prevention service, and collect identification information (for example, IMEI) of a mobile terminal 30 of the service user and information of a mobile communication system 20 to which the service user subscribed. Here, the consent to collection and use of personal information may include consent to information related to the purpose of collection and use of personal information (for example, providing an advice service about visit to an infectious disease-affected area), information to use (for example, wireless communication information between the terminal and a base station, collected by the mobile communication company), the period of collection and use of personal information (for example, equal to the incubation period of the occurred infectious disease), and personal information deletion (for example, deleting when cancelling the service).

In S83, the infectious disease proliferation prevention system 10 may transmit the infectious disease information and the consent to collection and use of personal information to a plurality of mobile communication systems 20, and receive affected area visitor terminal information from the plurality of mobile communication systems 20.

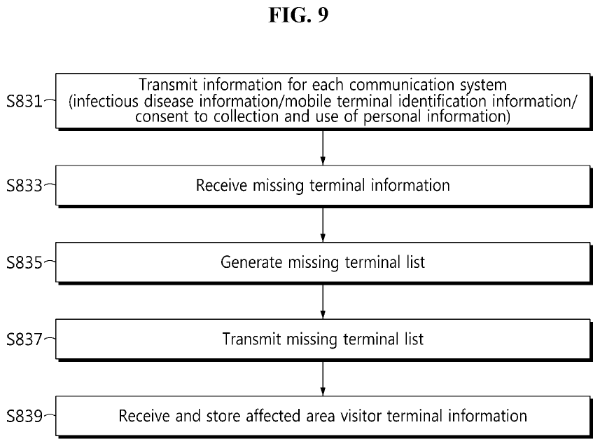

FIG. 9 is a detailed flowchart illustrating the step of receiving affected area visitor terminal information of FIG. 8. Referring to FIG. 9, the step of receiving affected area visitor terminal information (S83) may include transmitting information for each mobile communication system (S831), receiving missing terminal information (S833), generating a missing terminal list (S835), transmitting the missing terminal list (S837), and storing affected area visitor terminal information (S839).

In S831, the infectious disease proliferation prevention system 10 identifies the mobile communication systems 20 to which the plurality of service users subscribed by referencing the database 40, and classifies the service users for each mobile communication system 20. Additionally, the infectious disease proliferation prevention system 10 may transmit infectious disease information, identification information (for example, IMEI) of the mobile terminals 30 of the service users and consent to collection and use of personal information for each mobile communication system 20. Each of the plurality of mobile communication system 20 having received the consent to collection and use of personal information from the infectious disease proliferation prevention system 10 may extract mobile terminals 30 having consented to collect and use personal information from the mobile terminals 30 subscribed to the corresponding mobile communication system 20, and determine, as missing terminals, mobile terminals 30 that have not connected to the corresponding network for a preset period or have an international roaming service history among the extracted mobile terminals 30.

Referring to FIGS. 3 and 4, for example, the first mobile communication system 1 may determine, as missing terminals 12, 15, 17, 18, mobile terminals 30 that have not connected to the corresponding network for the preset period or have an international roaming service history among the plurality of mobile terminals 11, 12, 13, . . . , 1N received from the infectious disease proliferation prevention system 10. The second mobile communication system 2 and the Nth mobile communication system N may determine missing terminals 21, 22, 25, 26 and missing terminals N1, N2, N3 respectively by the same method. Subsequently, the first, second and Nth mobile communication systems 1, 2, N may transmit missing terminal information including identification information (for example, IMEI) of the missing terminals to the infectious disease proliferation prevention system 10.

In S833, the infectious disease proliferation prevention system 10 may receive the missing terminal information from each mobile communication system 20. According to an embodiment, the infectious disease proliferation prevention system 10 may receive the missing terminal information from the plurality of mobile communication systems 20 in the world in a preset cycle and store the missing terminal information in the database 40.

In S835, the infectious disease proliferation prevention system 10 may collect the missing terminal information received from each mobile communication system 20, and generate a missing terminal list. For example, as shown in FIG. 4, the infectious disease proliferation prevention system 10 may individually receive the missing terminal information from the first, second and Nth mobile communication systems 1, 2, N, collect the missing terminal information, and generate the missing terminal list shown in FIG. 5.

In S837, the infectious disease proliferation prevention system 10 may transmit the missing terminal list to the plurality of mobile communication systems 20. According to an embodiment, the infectious disease proliferation prevention system 10 may transmit the missing terminal list to the plurality of mobile communication systems 20 in the preset cycle. According to another embodiment, when the infectious disease proliferation prevention system 10 receives a missing terminal list request from any of the mobile communication systems 20, the infectious disease proliferation prevention system 10 may individually transmit the missing terminal list to the mobile communication system 20 having transmitted the request. Each of the plurality of mobile communication systems 20 having received the infectious disease information may extract base stations installed in affected areas by matching information about the affected areas with information of the base stations managed by the corresponding company, and extract mobile terminals 30 having a history of communication with the extracted base station. Each of the plurality of mobile communication systems 20 may determine mobile terminals 30 included in the missing terminal list among the extracted mobile terminals as affected area visitor terminals, and transmit the affected area visitor terminal information including identification information (for example, IMEI) of the affected area visitor terminal, the visited infectious disease-affected area, the name of the infectious disease and the period of stay to the infectious disease proliferation prevention system 10. For example, FIG. 6 shows an example of the affected area visitor terminal information transmitted to the infectious disease proliferation prevention system 10 by the mobile communication systems 1, 2, N.

In S839, the infectious disease proliferation prevention system 10 may receive the affected area visitor terminal information from the plurality of mobile communication systems 20 and store the affected area visitor terminal information in the database 40.

Referring back to FIG. 8, in S85, the infectious disease proliferation prevention system 10 may classify the affected area visitor terminal information received from the plurality of mobile communication systems 20 as described with reference to FIG. 9 by infectious disease, generate an affected area visitor terminal list by infectious disease, and store the affected area visitor terminal list in the database 40. For example, referring to FIGS. 6 and 7, the infectious disease proliferation prevention system 10 may collect the affected area visitor terminal information transmitted to the infectious disease proliferation prevention system 10 by the mobile communication systems 1, 2, N as shown in FIG. 6 and generate the affected area visitor terminal list by infectious disease as shown in FIG. 7. Here, the affected area visitor terminal list may include the items of identification information (for example, IMEI) of the affected area visitor terminal, the name of the infectious disease, the first visit date, the last visit date and the number of days left until the incubation period expires.

In S87, the infectious disease proliferation prevention system 10 may manage the affected area visitor terminal list by adding, updating and deleting the affected area visitor terminal information transmitted from the plurality of mobile communication systems 20 in the predetermined cycle. When the affected area visitor terminal is transmitted for the first time and thus there is no record stored in the database 40, the infectious disease proliferation prevention system 10 may add and store the corresponding the affected area visitor terminal information to the affected area visitor terminal list. In this instance, when the affected area visitor terminal is added to the affected area visitor terminal list for the first time, the infectious disease proliferation prevention system 10 may transmit the fact that the affected area visitor terminal entered the affected area to the affected area visitor terminal. According to an embodiment, the infectious disease proliferation prevention system 10 may transmit an infectious disease related message about the name of the infectious disease occurred in the affected area, symptoms of the corresponding infectious disease and actions to be taken when symptoms develop to the affected area visitor terminal in the form of an SMS message or an MMS message. Additionally, when there is a record of the affected area visitor terminal stored in the database 40, first, the infectious disease proliferation prevention system 10 may identify if the infectious disease name is the same, and when the infectious disease name is the same, may update the affected area visitor terminal list by changing the last visit date to the recently received date and re-calculating the number of days left until the incubation period expires. In this instance, the infectious disease proliferation prevention system 10 may transmit information about the corresponding infectious disease to the affected area visitor terminal in the form of an SMS message or an MMS message within the incubation period. When the infectious disease name is different for the same affected area visitor terminal, the infectious disease proliferation prevention system 10 may determine the exposure to a new infectious disease, generate data together with the name of the infectious disease and add the data to the affected area visitor terminal list for the corresponding infectious disease. Additionally, the infectious disease proliferation prevention system 10 may update the affected area visitor terminal list in the predetermined cycle, and when the affected area visitor terminal list is updated, may calculate affected area visitor terminal information in which the incubation period is determined to expire and delete the affected area visitor terminal information from the affected area visitor terminal list. For example, FIG. 7 shows calculation of affected area visitor terminal information in which the incubation period is determined to expire by the infectious disease proliferation prevention system 10 under the assumption that the current date is Mar. 2, 2017. In the case of the mobile terminal 17 of FIG. 7, when a result (for example, Feb. 24, 2017) of adding the final visit date (for example, Feb. 10, 2017) to the incubation period (14 days) in the recently updated affected area visitor terminal list is earlier than the current date (Mar. 2, 2017), the infectious disease proliferation prevention system 10 may determine that the incubation period expired (the number days left until the incubation period expires=0), and delete the mobile terminal 17 from the affected area visitor terminal list. When the affected area visitor terminal is deleted from the affected area visitor terminal list, the infectious disease proliferation prevention system 10 may inform the expiration of the incubation period to the affected area visitor terminal in the form of an SMS message or an MMS message.

While this specification contains many features, the features should not be construed as limiting the scope of the disclosure or the appended claims. Certain features described in the context of separate exemplary embodiments can also be implemented in combination in a single exemplary embodiment. Conversely, various features described in the context of a single exemplary embodiment can also be implemented in multiple exemplary embodiments separately or in any suitable subcombination.

Although the drawings describe the operations in a specific order, it should not be understood that the operations are performed in the specific order as shown in the drawings or in a sequential order, or all the operations are performed to obtain a desired result. Multitasking or parallel processing may be advantageous under a particular environment. Moreover, it should be understood that all exemplary embodiments do not require the distinction of various system components made in the above-described embodiments. The program components and systems may be generally implemented as a single software product or multiple software product packages.

The above-described method of the present disclosure may be implemented in program and recorded in a computer-readable recording medium (such as, for example, a compact disk-read only memory (CD ROM), random access memory (RAM), read-only memory (ROM), floppy disk, hard disk, magneto-optical disk, and the like). This process may be easily performed by person having ordinary skill in the technical field to which the present disclosure belongs, and its detailed description is omitted herein.

It should be noted various substitutions, modifications, and changes may be made to the present disclosure by person having ordinary skill in the technical field to which the present disclosure belongs without departing from the scope of the present disclosure, and the present disclosure is not limited by the above-described embodiments and the accompanying drawings.

What is claimed is:

1. A system for preventing the spread of an infectious disease, comprising:
at least one processor; and
a memory including at least one command that the at least one processor can execute,
wherein the at least one processor is configured to:
collect infectious disease information,
receive consent to collection and use of personal information from a service user who wants to use a service for preventing the spread of an infectious disease,
transmit the infectious disease information and the consent to collection and use of personal information to a plurality of mobile communication systems, and receive, from the plurality of mobile communication systems, information of a mobile terminal having visited an affected area, wherein the information of a mobile terminal is calculated based on the infectious disease information, the consent to collection and use of personal information and wireless communication information of the mobile terminal, and
classify and manage the information on the mobile terminal having visited the affected area received from the plurality of mobile communication systems by infectious disease.

2. The system according to claim 1, wherein the at least one processor is configured to:
receive missing terminal information based on the consent to collection and use of personal information from each of the plurality of mobile communication systems, collect the missing terminal information and generate a missing terminal list, and
transmit the missing terminal list to the plurality of mobile communication systems, and receive the information of the mobile terminal having visited the affected area from the plurality of mobile communication systems in response.

3. The system according to claim 2, wherein the missing terminal information is information about a mobile terminal that has consented to collect and use personal information, but has not connected to a corresponding network for a preset period or has an international roaming service history among mobile terminals subscribed to each mobile communication system.

4. The system according to claim 2, wherein the information of the mobile terminal having visited the affected area is information about a mobile terminal that has a history of communication with a base station that matches the infectious disease information, and is included in the missing terminal list.

5. The system according to claim 2, wherein the at least one processor classifies the information of the mobile terminal having visited the affected area, received from the plurality of mobile communication systems by infectious disease, and generates and stores a list,
wherein the list classified by infectious disease includes at least one of identification information of the mobile terminal having visited the affected area, an infectious disease name, a first visit date, a last visit date, and number of days left for an incubation period expires.

6. The system according to claim 5, wherein each time the at least one processor receives the information of the mobile terminal having visited the affected area from the plurality of mobile communication systems, the at least one processor updates the last visit date and the number of days left until the incubation period expires for the corresponding mobile terminal in the list classified by infectious disease.

7. The system according to claim 6, wherein the at least one processor transmits a message notifying expiration of the incubation period of the infectious disease to the mobile terminal with the expired incubation period of the infectious disease based on the list classified by infectious disease.

8. The system according to claim 2, wherein the at least one processor transmits the infectious disease information, and the consent to collection and use of personal information of a service user having subscribed to a corresponding mobile communication system, except a service user having subscribed to other mobile communication system, to each of the plurality of mobile communication systems.

9. The system according to claim 1, wherein the infectious disease information includes at least one of an infectious disease name, date of infectious disease outbreak, date of infectious disease eradication, a name of an affected country, and a name of an affected area.

10. The system according to claim 1, wherein the at least one processor transmits a message notifying an entry into the affected area to the mobile terminal having visited the affected area.

11. A method for preventing the spread of an infectious disease by a system for preventing the spread of an infectious disease, the method comprising:
collecting infectious disease information;
receiving consent to collection and use of personal information from a service user who wants to use a service for preventing the spread of an infectious disease;
transmitting the infectious disease information and the consent to collection and use of personal information to a plurality of mobile communication systems;
receiving information of a mobile terminal having visited an affected area, wherein the information of a mobile terminal is calculated based on the infectious disease information, the consent to collection and use of personal information and wireless communication information of the mobile terminal from the plurality of mobile communication systems; and
classifying and managing the information of the mobile terminal having visited the affected area, received from the plurality of mobile communication companies by infectious disease.

12. The method according to claim 11, wherein receiving the information of the mobile terminal having visited the affected area comprises:
receiving missing terminal information based on the consent to collection and use of personal information from each of the plurality of mobile communication systems, and collecting the missing terminal information and generating a missing terminal list; and
transmitting the missing terminal list to the plurality of mobile communication systems, and receiving the information of the mobile terminal having visited the affected area from the plurality of mobile communication systems in response.

13. The method according to claim 12, wherein the missing terminal information is information about a mobile terminal that has consented to collection and use of personal information among mobile terminals subscribed to each mobile communication system, but has not connected to a corresponding network for a preset period or has an international roaming service history.

14. The method according to claim 12, wherein the information of the mobile terminal having visited the affected area is information of a mobile terminal that has a history of communication with a base station matching the infectious disease information and is included in the missing terminal list.

15. The method according to claim 12, wherein classifying and managing comprises classifying the information of the mobile terminal having visited the affected area received from the plurality of mobile communication systems by infectious disease and generating and storing a list,
wherein the list classified by infectious disease includes at least one of identification information of the mobile terminal having visited the affected area, an infectious disease name, a first visit date, a last visit date, and number of days left until an incubation period expires.

16. The method according to claim 15, further comprising:
updating the last visit date and the number of days left until the incubation period expires for the corresponding mobile terminal in the list classified by infectious disease, each time the information of the mobile terminal having visited the affected area is received from the plurality of mobile communication systems.

17. The method according to claim 16, further comprising:
transmitting a message notifying expiration of the incubation period of the infectious disease to the mobile terminal with the expired incubation period of the infectious disease based on the list classified by infectious disease.

18. The method according to claim 12, wherein transmitting the consent to collection and use of personal information comprises transmitting the infectious disease information and the consent to collection and use of personal information of a service user having subscribed to a corresponding mobile communication system, except a service user having subscribed to other mobile communication system, to each of the plurality of mobile communication systems.

19. The method according to claim 11, wherein the infectious disease information includes at least one of an infectious disease name, date of infectious disease outbreak, date of infectious disease eradication, a name of an affected country, and a name of an affected area.

20. The method according to claim 11, further comprising:
transmitting a message notifying an entry into the affected area to the mobile terminal having visited the affected area.
